(19) United States
(12) Patent Application Publication
(10) Pub. No.: US 2023/0375723 A1
(43) Pub. Date: Nov. 23, 2023

(54) GAS SENSOR

(71) Applicant: Airthings ASA, Oslo (NO)

(72) Inventor: Bjørn Magne SUNDAL, Oslo (NO)

(73) Assignee: Airthings ASA, Oslo (NO)

(21) Appl. No.: 18/027,360

(22) PCT Filed: Sep. 21, 2021
(86) PCT No.: PCT/EP2021/075960

§ 371 Date: Mar. 20, 2023

(30) Foreign Application Priority Data
Sep. 22, 2020 (GB) 2014952.2

(51) Int. Cl. G01T 1/178; G01N 33/00; H05K 1/18

(52) U.S. Cl. CPC G01T 1/178; G01N 33/0055; H05K 1/183; H05K 2201/10106; H05K 2201/10545

(57) ABSTRACT

A sensor, comprising: a printed circuit board; a detector mounted on the printed circuit board; an inner dome that is electrically conductive and is mounted on the printed circuit board so as to form a diffusion chamber around the detector; and an outer dome that is electrically conductive and surrounding the inner dome. The dual dome construction allows a stronger electric field to be generated inside the inner dome. The strength of the electric field is determined by the voltage of the detector, the voltage of the inner dome and the distance between them. The detector has a maximum voltage that can safely be applied to it without damaging the detector. With the dual dome design, the inner dome can be biased to a higher potential, thereby increasing the strength of the electric field inside the inner dome, while still shielding that high voltage via the outer dome.

The present invention relates to gas sensors. Preferred embodiments relate to radon gas sensors.

Radon is a radioactive element which at normal temperature and pressure is a gas. It is colourless, odourless and tasteless which means that its presence and concentration is not readily detectable by human beings. However, due to its radioactivity, it can be harmful if the concentration is too high. At normal concentrations, radiation from radon typically accounts for around half of a person's annual natural radiation dose.

The most stable isotope of radon is radon-222 which has a half life of 3.8 days and is produced as part of the decay chain of uranium-238 which is present throughout the Earth's crust. Being a noble gas, radon readily diffuses out of the ground and into the air around us. The daughter products of radon decay tend to be charged particles which will readily stick to dust or smoke particles in the air. When these particles are inhaled, they can lodge in the lungs and the subsequent radiation from decay of the radon daughter products causes a risk of lung cancer. Consequently, higher concentrations of radon lead to higher risks of cancer.

The concentration of radon in the atmosphere depends, amongst other things, on ventilation. Areas with good ventilation will have lower radon concentrations, whereas a lack of ventilation leads to radon accumulation and thus increases the radiation level in such areas. Radon levels outside therefore tend to be lower than inside buildings. For example, typical radiation doses from radon may be around 10-20 Bq/m³ outside and may be around 100 Bq/m³ inside. Radon levels can also vary significantly due to variations in geographic location (e.g. different geologies), or due to differences in building materials.

Radon decays by emission of an alpha particle with an energy of 5.5 MeV. The resultant Polonium-218 has a half life of about 3 minutes before emitting an alpha particle of 6.0 MeV. The resultant Lead-214 has a half life of around 27 minutes before beta-decaying to Bismuth-214 which in turn has a half life of 20 minutes and beta-decays to Polonium-214. Polonium-214 has a half life of about 164 microseconds before emitting an alpha particle of 7.7 MeV resulting in Lead-210 which has a half life of 22 years and is thus relatively stable.

Detection of radon to date has been divided into two main methods. The first method is active detection of alpha particles using a photodiode and the second method is passive detection of alpha particles using a track detector. Typically the first method requires a large instrument and needs electrical power to be supplied. Such instruments have typically only been used for larger scale, e.g. commercial or industrial measurements as the instruments are more bulky and expensive. The photodiode (e.g. a PIN diode) is placed in a diffusion chamber of the device. Alpha particles hitting the photodiode create a number of electron-hole pairs which will cause a small current to be generated. These current signals can be detected and counted to provide a measure of the radon concentration within the diffusion chamber. Such active measurements can be provided continuously in time rather than having to wait for the results of a laboratory analysis.

The second method uses much smaller detectors with no power requirement and is thus much more suited to domestic customers. A passive (i.e. unpowered) track chamber is typically placed in a selected location and left for a predetermined period of time (typically from a few weeks and up to about 3 months) after which it is sent back to a lab for analysis. Alpha particles emitted within the chamber leave tracks on a film which is also disposed within the chamber. These tracks can be detected in the lab and counted thus providing a measure of the radon concentration in the air within the chamber.

WO 2008/080753 describes a passive radon detector device with a diffusion chamber rotatably mounted above the detector so that it can be rotated in and out of the “ON” position above the detector. When the chamber is in position above the detector, the detector will detect alpha particles from gas which diffuses into the chamber. When the chamber is rotated out of position (the “OFF” position), the detector is covered (the chamber volume is essentially reduced to zero) and is therefore effectively isolated from radon in the surrounding environment.

US 2009/0230305 describes an active radon detector device which is battery powered. The photodiode detector is mounted on the main PCB and is covered by a sampling chamber, also mounted on the main PCB. Air enters and leaves the sampling chamber through apertures in the PCB. These apertures are optionally covered by a filter to exclude undesired debris such as smoke, dust, and the like.

U.S. Pat. No. 5,489,780 describes another active radon detector device in which a pressed metal filter is used as the wall of the diffusion chamber. This filter is mounted directly on the PCB over the photodiode detector, thus defining the sampling volume.

According to the present invention there is provided a sensor, comprising:
a printed circuit board;
a detector mounted on the printed circuit board;
an inner dome that is electrically conductive and is mounted on the printed circuit board so as to form a diffusion chamber around the detector; and
an outer dome that is electrically conductive and is mounted on the printed circuit board, surrounding the inner dome.

The dual dome construction allows a stronger electric field to be generated inside the inner dome, i.e. between the inner dome and the detector. The strength of the electric field is determined by the voltage of the detector, the voltage of the inner dome and the distance between them. Therefore, for a given size/shape of diffusion chamber (i.e. a given size/shape of inner dome), the relative voltages determine the electric field strength. The detector normally has a maximum voltage that can safely be applied to it without damaging the detector. For example, in some examples, the detector may be negatively biased by up to −70 V without damage. In previous sensors, the dome has been held at ground potential so that the voltage difference between the dome and the sensor in this example would by 70 V. However, with the dual dome design, the inner dome can now be biased to a much higher potential, thereby increasing the strength of the electric field inside the inner dome, while still shielding that high voltage via the outer dome. The outer dome is preferably at ground potential for several reasons. One reason is for safety; by having it at ground potential there is less risk of electrical shock when the module is operated without a protective instrument housing. Another important reason is to provide electrical shielding to the inner dome. The high voltage put on the inner dome is for reasons of power efficiency (the module is designed for extreme low power operation in order to support long life time on batteries) supplied by a high-voltage generator with a high output impedance. This high voltage is susceptible to electromagnetic pick-up and in order to avoid such pick-up the outer dome provides an electromagnetic shielding of the inner dome. In addition, the potential difference between the inner dome and the outer dome generates an electric field which encourages “plate out” of charged radon daughters or aerosols in the diffusion path between the two domes.

In a radon sensor, the strength of the electric field is important for sensitivity as it affects the ability of the sensor to collect charged radon daughter products (also referred to as “progenies”). When a Radon atom decays into a Polonium-218 atom, the Polonium atom is normally positively charged and can thereby be drawn by the electric field onto the surface of the photodiode. Once landed on the photodiode, there is a 50% chance of any subsequent decay hitting the photodiode and generating a signal. However, the charged Polonium atoms lose their charge quickly through collisions with other air molecules by which they can pick up electrons. Once neutralised, the electric field no longer has any effect. Therefore it is desirable to accelerate the charged daughter products towards the detector surface as fast as possible. This is achieved with as high a field strength as possible. With the arrangements described here, it is possible to raise the potential of the inner dome to a high voltage, thereby increasing the voltage difference between the inner dome and the detector, while still shielding that high voltage from the outside. For example, the inner dome could be held at a potential of at least 30 V, or at least 50 V or at least 70 V or at least 100 V. With a potential of 100 V on the inner dome and a potential of −70 V on the detector, a voltage difference of 170 V can be achieved, with a correspondingly strong electric field between them.

The dual dome design also has other benefits. For example, it can be used to form at least part of the diffusion path, which is an important characteristic of a diffusion chamber. The diffusion path determines the rate at which air can enter and leave the sensitive volume of the chamber (the inner dome). The diffusion path provides a restrictive path that air must follow in order to reach the diffusion chamber. This path can be used to influence the characteristics of the air entering the diffusion chamber. For example, in a radon sensor, the diffusion path can be used to reduce the chance of radon daughter products entering the chamber. Radon is a noble gas with a half life of 3.8 days and therefore does not interact with the diffusion path. It can therefore pass freely along the diffusion path. By contrast, the Polonium daughter products of Radon or typically charged and therefore have a strong tendency to attach to trace gases such as water vapour and/or to larger aerosols (e.g. dust particles) which then readily stick to nearby surfaces. They also have much shorter half lives. Therefore a narrow diffusion path with a time constant comparable with or greater than the half life of Polonium-218 has a good chance of capturing the Radon daughter products (whether charged or uncharged) on the surfaces of the path and preventing them from reaching the sensitive inner volume. This ensures that any alpha particle disintegrations detected within the inner volume can be assumed to have originated from a Radon molecule within the sensitive volume. The dual dome design creates a space between the outer surface of the inner dome and the inner surface of the outer dome that can be used as part of the diffusion path. When this is combined with different voltages on the inner and outer domes (e.g. a high voltage on the inner dome and a lower voltage on the outer dome), an electric field across the diffusion path further encourages charged daughter products to “plate out” on the walls of the diffusion path.

It will be appreciated that the term “dome” is used here in a general sense to mean any inverted bowl shape. It is not limited to hemispherical domes, but rather also includes cylindrical domes, cuboid domes, pyramidal domes. When mounted adjacent to the printed circuit board, the inner dome defines a chamber with the detector inside the chamber and the chamber defines a sensitive volume which may be of relevance to the detector (for example in a radon sensor it determines the volume of gas from which radon disintegrations may be detected).

The outer dome surrounds the inner dome in the sense that it surrounds it on all sides except that of the printed circuit board. Thus the inner dome lies within the interior volume of the outer dome.

As discussed above, the sensor may be arranged to apply a first voltage to the inner dome and a second voltage to the outer dome. The second voltage is preferably different to the first voltage so as to create an electric field between the inner dome and the outer dome to encourage charged particles to accelerate towards and subsequently attach to one of the domes. The first voltage at least partly defines the strength of an electric field inside the inner dome.

It will be appreciated that a voltage difference can be generated between the two domes by applying a higher voltage to the outer dome than the inner dome. However, it is generally preferred to keep the voltage of the outer dome closer to zero volts or even at zero volts (ground). Therefore in some embodiments the first voltage has a magnitude greater than that of the second voltage. It will be appreciated that where the first voltage is positive, the second voltage is less positive and if the first voltage is negative, the second voltage is less negative. In preferred embodiments the first voltage is positive as the detector tends to be most suitable for negative biasing (so a positively biased inner dome creates the strongest electric field).

The second voltage may be ground so as to minimise electric fields outside of the outer dome.

The sensor may be arranged to apply a detector bias voltage to the detector. The detector bias voltage may be different to both the first and second voltages. As discussed above, the detector bias voltage is ideally of the opposite sign to the first voltage so as to maximise the electric field strength between the inner dome and the detector.

The inner dome may be connected to a first conductive layer of the printed circuit board so as to form a faraday shield around the detector. The first conductive layer may extend inwardly from the rim of the inner dome towards the detector so as to form a substantially continuous conductive plane on top of the printed circuit board and around the detector. A gap may be formed around the detector so as to isolate the detector from the first conductive layer and the inner dome (so that it can be biased to a different potential). The first conductive layer may be a surface conductive layer of the printed circuit board which may be a multilayer printed circuit board. The first conductive layer together with the inner dome form the faraday shield that substantially surrounds the detector and protects it from electromagnetic interference.

The outer dome may be connected to a second conductive layer of the printed circuit board so as to form a faraday shield around the inner dome. The second conductive layer may be a surface layer on the opposite side of the printed circuit board, but is preferably an internal layer of a multilayer printed circuit board. The internal layer can pass underneath the first conductive layer that connects to the inner dome and thereby encase the whole of the faraday shield of the inner dome within a second outer faraday shield formed by the second conductive layer and the outer dome. It will be appreciated that connections to the detector and the inner dome (and the first conductive layer) must be made through the second conductive layer, but these only require small holes to be made in the second conductive layer so that an electrical via can pass through to make a connection inside the outer faraday shield. This will not significantly impair the effectiveness of the outer faraday shield. Other electrical components may be mounted to a third conductive layer of the printed circuit board on the opposite side to that of the domes. These components (and the circuits that they form) will be completely shielded from the high voltage applied to the inner dome and the first conductive layer by the interposing second conductive layer.

The inner dome and the outer dome need not be symmetrically arranged. They also do not need to be the same shape. For example, the inner dome may be offset within the outer dome so that it lies closer to one side thereof than another. Similarly, the outer dome could be a hemisphere while the inner dome is cylindrical. However, in many cases it may be preferred for various reasons to have symmetry of some sort. Therefore the inner dome and the outer dome may be substantially the same shape and concentrically arranged. In such cases the main shape of the outer dome is simply a larger version of the shape of the inner dome and the two domes may be arranged such that there is a uniform gap between them all the way around, i.e. the gap between the inner dome and the outer dome is substantially the same all round the inner dome. It will be appreciated that slight variations may occur due to the particular choice of shape. For example two cuboid domes will have a slightly larger distance from the corner of the inner dome to the corner of the outer dome then they will between the centre of an inner face and the centre of an outer face (simply due to the geometry), but the gap will still be substantially equal around most of the area of the inner dome. In the case of a radon gas sensor, the uniform spacing can be used as part of a substantially uniform diffusion path. The uniformity makes it easier to calculate the probabilities of decays with high certainty.

The shape of the inner dome in particular influences the electric field that can be set up inside the inner dome. Sharp corners result in a weaker electric field and therefore it is preferred that the inner dome has a rounded shape, i.e. one without sharp corners or edges. Rounding the corners of the inner dome increases the uniformity of the electric field and also increases the uniformity of the gap between inner and outer domes when they are concentrically arranged. In certain preferred embodiments the inner dome has a rounded cuboid shape with rounded edges and corners. It will be appreciated that the outer dome may also have the same rounded cuboid shape with rounded edges and corners (but slightly larger in size than the inner dome).

As discussed above, a diffusion path for air exchange with the interior volume of the inner dome may pass between the inner dome and the outer dome. The diffusion path may comprise some or all of the intervening space between the two domes, but in some embodiments an entrance to the diffusion path may be located centrally in a roof of the outer dome. Locating the entrance centrally on the roof has the advantage of symmetry, i.e. that the diffusion path length from the central entrance will be more or less the same in all directions away from the entrance (assuming the entrance into the inner dome is also symmetrically arranged with respect to the entrance in the outer dome). The entrance in the roof of the outer dome may comprise one or more holes formed through the outer dome.

In some embodiments the hole may simply allow air to flow unimpeded through the hole in the outer dome. However, in other embodiments, a filter (e.g. filter paper) may be provided over the entrance so as to filter out dust particles from entering the diffusion path. The filter paper may be the same as is used in standard air filters, although it may be noted that there is an additional benefit of the filter paper not requiring regular replacement as there is no pressure difference across the paper and therefore no significant build up of particles to clog the pores of the filter paper.

In order to ensure that the diffusion path extends along the gap between the inner and outer chambers, it is necessary to ensure that it is not bypassed or shortened by an alternative air path underneath the rim of the outer dome, adjacent to the printed circuit board. This could be achieved by applying a sealant around the join between the outer dome and the printed circuit board. However, such a sealant is messy and semi-permanent and adds a step to the assembly process. Therefore in some preferred embodiments the sensor further comprises a gasket arranged to seal against a surface of the printed circuit board. The seal provided by the gasket blocks air flow underneath the gasket, i.e. between the gasket and the printed circuit board. The seal provided by the gasket also blocks light from entering under the domes adjacent to the printed circuit board. This is important in embodiments where the detector is a photosensor such as a photodiode (e.g. PIN diode) or photomultiplier (e.g. Silicon photomultiplier) which are sensitive to light. A photodiode is often used in a radon sensor as it is sensitive to alpha particles, but it remains sensitive to light and so a light sealed diffusion chamber is still important. For this reason, the gasket is also preferably a dark colour, e.g. black to absorb light.

In order to create a good seal against the printed circuit board, the gasket is ideally biased against the printed circuit board. Any structure for doing so may be used, but it is convenient if that structure is provided by one of the domes. Therefore in some embodiments the gasket may be biased against the printed circuit board by a lip formed on at least one of the inner dome and the outer dome.

The lip could be formed on the inside surface of the inner dome, with the gasket also then being located on the inside of the inner dome. However, this is not particularly convenient in embodiments which rely on creating an electric field as the presence of the gasket interferes with the uniformity and strength of the electric field. The lip could instead be formed on the outside of the outer dome, with the gasket also then situated around the outer circumference of the outer dome. However, this increases the overall area of the device and the size of the gasket. Thus, in certain preferred embodiments, the gasket is located between the inner dome and the outer dome. The lip that holds the gasket in place may be formed on the outer surface of the inner dome or the inner surface of the outer dome, or partly on each.

The gasket preferably seals against an inner surface of the outer dome. Such sealing, together with the sealing against the printed circuit board prevents air from entering the space between the inner dome and the outer dome underneath the rim of the outer dome. The vertical path over the gasket is blocked by the seal between the gasket and the inner surface of the outer dome and the horizontal path underneath the gasket is blocked by the seal between the gasket and the printed circuit board. Therefore the space between the inner dome and the outer dome is only accessible by other deliberately formed entrances that may be used as part of creating a diffusion path as discussed above.

The diffusion path may enter the inner dome in any way. For example, holes could be provided through the side walls of the inner dome at selected places. However, in order to maintain the uniformity of the inner dome walls for uniformity of electric field creation, it is preferred that air enters the inner dome underneath the rim of the inner dome. With the gasket located between the inner dome and the outer dome, the gasket may be in contact with both. It is convenient for assembly that the gasket is in contact with the outer surface of the inner dome so that the gasket can be assembled onto the inner dome by wrapping around the inner dome. The gasket may then remain in place just through friction with the inner dome or with the assistance of a slight stretch as it is placed onto the inner dome. As the gasket preferably forms a seal against the printed circuit board, air that enters the inner dome in this arrangement must pass over the gasket and between the gasket and the inner dome. Accordingly, in some embodiments the gasket seals against an outer surface of the inner dome except that one or more air channels are formed to bypass the gasket and are formed along the outer surface of the inner dome, connecting with a rim of the inner dome adjacent to the printed circuit board. Air can therefore pass along these channels, behind the gasket, thereby reaching the rim of the inner dome.

In some embodiments the gasket is biased against the printed circuit board by a lip formed on the inner dome and the one or more air channels each extends along the underside of the lip. Forming the lip on the inner dome (rather than on the outer dome) is also convenient for mounting the gasket during the assembly process as it helps to hold the gasket in the correct position throughout the assembly process. The gasket can be pushed over the rim of the inner dome so as to be seated around the circumference of the inner dome and pressed up until it contacts the lip. As the gasket contacts the lip, the air channels extend along the underside of the lip so as to ensure air communication over the gasket and down to the rim of the inner dome.

In order to effect a good seal between the gasket and the printed circuit board, the gasket should be compressed slightly to as to ensure uniform contact with the printed circuit board around the whole length of the gasket. Where the gasket is also in contact with the inner dome and/or outer dome, the gasket can also be compressed against those surfaces to effect a good seal.

It is also desirable to ensure electrical connection of the inner dome and the outer dome with the printed circuit board so as to allow suitable voltages to be applied to them.

A biasing member may therefore be provided to bias the outer dome towards the printed circuit board and to ensure electrical contact of the outer dome with the printed circuit board. The biasing member could be any device or mechanism that provides a force on the outer dome that acts towards the printed circuit board. For example a strap could be applied over the dome and tensioned to pull it towards the printed circuit board. Alternatively, a structure from another component (e.g. an instrument housing) could be arranged to press on the top of the outer dome to press and hold it against the printed circuit board.

In some embodiments the biasing member comprises one or more clips provided on the outer dome that extend through holes in the printed circuit board and engage with a side of the printed circuit board opposite the side on which the outer dome is located. In embodiments where the biasing of the outer dome towards the printed circuit board results in compressing a gasket, the gasket provides a reaction force that biases the outer dome away from the printed circuit board. This reaction force can lift the outer dome away from the printed circuit board preventing electrical contact from being made between the printed circuit board and the rim of the outer dome. Therefore in some embodiments the biasing member is arranged to provide the electrical connection between the outer dome and the printed circuit board. In the case of the clips discussed above, the reaction force from the gasket biases the clips against the opposite side of the printed circuit board. Therefore the clips may be electrically conductive and arranged to contact a conductive layer on the opposite side of the printed circuit board, thereby providing the electrical connection between the outer dome and the printed circuit board.

It is also desirable to bias the inner dome into electrical contact with the printed circuit board. This may be done by providing an inner dome biasing member that could be a strap or clips as discussed above. However, it is also desirable to avoid significant structure on the printed circuit board within the gap between the inner dome and the outer dome, especially as this is where the gasket may be located. In some embodiments the outer dome is arranged to bias the inner dome into electrical contact with the printed circuit board. When the outer dome is itself biased towards the printed circuit board, it can be used to transmit biasing force to the inner dome, thereby biasing the inner dome towards the printed circuit board. Such biasing could be achieved by one or more integral projections formed on an inner surface of the outer dome and arranged to project towards the printed circuit board and contact the outer surface of the inner dome. However, in some embodiments it is preferred to keep the moulding of the outer dome simple and therefore a separate biasing member may be provided to transmit force to the inner dome. For example a spring can be provided between the inner dome and the outer dome to transmit the biasing force from the outer dome onto the inner dome.

A spring acts to accommodate some relative movement between the inner and outer dome so as to allow both domes to contact the printed circuit board while also transmitting the biasing force from the outer dome to the inner dome. However, in some embodiments the roof of the inner dome and/or the roof of the outer dome is sufficiently flexible to accommodate such relative movement. In such cases a separate spring is not required. Therefore in some examples a spacer is provided between the outer dome and the inner dome so as to transmit a biasing force from the outer dome to the inner dome. The spacer need not be elastic, i.e. it may be substantially rigid.

As discussed above, an entrance to a diffusion path may be located centrally in a roof of the outer dome. The spacer may form a ring around the entrance and may have one or more holes or channels formed in its side wall to allow air to flow from the entrance along the diffusion path. When the spacer surrounds the entrance it provides an obstruction to the diffusion path and therefore it is necessary to ensure that air can flow past it to continue along the diffusion path. The spacer and the holes or channels may be symmetrical to maintain symmetry of the diffusion path.

As discussed above, a filter may be provided adjacent to the entrance. In such embodiments the filter (e.g. filter paper) may be held adjacent to the opening by the spacer. The filter may be interposed between the spacer and the outer dome.

As part of the assembly process of mounting the inner dome and the outer dome to the printed circuit board, it is necessary to align the inner dome and the outer dome with electrical contacts on the printed circuit board. This can be achieved by any form of locating structure such as ridges or grooves in the printed circuit board. However, in some embodiments one of the inner dome and the outer dome comprises one or more locating pins extending towards the printed circuit board and the printed circuit board has a corresponding one or more locating recesses formed therein to receive the one or more locating pins, and wherein the one or more locating recesses are sufficiently deep that the one or more locating pins do not contact the bottom of the one or more recesses. Ensuring that the locating recesses are deep enough that the locating pins do not contact the bottoms of the recesses means that the locating pins cannot define the relative height of the dome and the printed circuit board. The proximity (or indeed contact) of the domes with the printed circuit board is important to ensure proper functioning of the device. For example, if the outer dome provides a biasing force on the inner dome, any restriction of its movement caused by locating pins contacting the bottom of locating recesses could reduce the force applied to the inner dome. For the inner dome, where the diffusion path is arranged to pass under the rim of the inner dome, any contact between the locating pins and the bottom of locating recesses could alter the gap under the rim, changing the diffusion path properties. If such contact between locating recesses and locating pins were to be allowed then the manufacturing tolerances of the pins and recesses would have to be very precise which would add to the costs. Instead, ensuring that the recesses are deep enough to avoid such contact means that they can provide the locating function without having to be a precise depth.

A single locating pin can be used to achieve alignment both in terms of position and angle, e.g. if the pin is shaped to allow mating in only one orientation (e.g. an elongate pin or a square pin). However, for simplicity of manufacture, rounded pins and rounded recesses are preferred and therefore to ensure both spatial and angular alignment it is preferred to provide two or more locating pins and two or more corresponding locating recesses.

In some embodiments the locating pins are formed on the inner dome. It is advantageous to have the locating pins on the inner dome as they can then be used to hold the inner dome (together with the gasket) in the correct position and orientation while a tool is used to mount the outer dome over the inner dome in order to seal the module.

In some embodiments the one or more locating pins are formed on the inner dome, the inner dome comprises one or more spacer projections formed on the rim and extending towards the printed circuit board, and the depth of each of the one or more recesses is greater than the difference between the length of the corresponding locating pin and the length of the spacer projections. The spacer projections are arranged to contact the printed circuit board so as to define the gap under the rim of the inner dome by which air can enter the inner dome. As both the spacer projections and the locating pins extend from the rim of the inner dome, it is the difference between their lengths that determines the depth that the recess must exceed.

In some embodiments the printed circuit board will be a multilayer printed circuit board with at least one surface conductive layer and at least one internal conductive layer. It will be appreciated that the depth of the locating recesses is likely to be greater than the distance between the surface conductive layer and the internal conductive layer. For example, a typical printed circuit board may have a distance of approximately 200 microns between such layers. Therefore in some embodiments the printed circuit board is a multilayer printed circuit board comprising a surface conductive layer, portions of which are in contact with the inner dome and the outer dome, and an internal conductive layer located at a first depth below the surface conductive layer, wherein the depth of the one or more locating recesses is greater than the first depth and wherein the internal conductive layer comprises an insulating region around each of the one or more locating recesses. The insulating regions ensure that no electrical contact is made between the internal conductive layer and the locating pins (which may be conductive as they are part of the conductive dome).

In some embodiments the one or more locating recesses is lined with electrically conductive material. Where the locating recesses extend through an internal conductive layer of a multilayer printed circuit board, this lining ensures a continuous faraday shield is formed within the locating recesses.

The arrangement for sealing against the printed circuit board is considered to be independently inventive and may be applicable to embodiments in which only a single dome is used. Therefore according to another aspect of the invention, there is provided a sensor, comprising:
a printed circuit board;
a detector mounted on the printed circuit board;
a dome that is electrically conductive and is mounted on the printed circuit board so as to form a diffusion chamber around the detector; and
a gasket arranged to seal against a surface of the printed circuit board wherein the gasket is biased against the printed circuit board by a lip formed on the dome.

As discussed above, the lip may extend from an outer surface of the dome. The gasket may seal against an outer surface of the dome except that one or more air channels are formed to bypass the gasket and are formed along the outer surface of the dome, connecting with a rim of the dome adjacent to the printed circuit board. The one or more air channels may each extends along the underside of the lip.

It will be appreciated that other preferred and optional features described above in relation to the dual dome design may also be applied here. For example a bias voltage may be applied, locating projections and recesses may be used, biasing members may be provided, etc.

In the single dome design, a diffusion path into the interior of the dome will of course not be defined by an outer dome, but other structure may be used to provide such a path, or it may simply rely on holes and channels provided in or under the rim of the dome and/or around the gasket.

The use of locating pins and recesses is also considered to be independently inventive. Therefore, according to another aspect of the invention there is provided a sensor, comprising:
a printed circuit board;
a detector mounted on the printed circuit board;
a dome that is electrically conductive and is mounted on the printed circuit board so as to form a diffusion chamber around the detector; and
wherein the dome comprises one or more locating pins extending towards the printed circuit board and wherein the printed circuit board has a corresponding one or more locating recesses formed therein to receive the one or more locating pins, and wherein the one or more locating recesses are deeper than the length of the one or more locating pins.

The dome may comprise one or more spacer projections formed on the rim and extending towards the printed circuit board, and wherein the depth of each of the one or more recesses is greater than the difference between the length of the corresponding locating pin and the length of the spacer projections.

The printed circuit board may be a multilayer printed circuit board comprising a surface conductive layer, portions of which are in contact with the dome, and an internal conductive layer located at a first depth below the surface conductive layer, wherein the depth of the one or more locating recesses is greater than the first depth and wherein the internal conductive layer comprises an insulating region around each of the one or more locating recesses.

Each of the one or more locating recesses may be lined with electrically conductive material.

It will be appreciated that other preferred and optional features described above in relation to the dual dome design may also be applied here. For example a bias voltage may be applied, a gasket may be used, biasing members may be provided, etc.

Certain preferred embodiments of the invention will now be described, by way of example only, and with reference to the accompanying drawings in which:

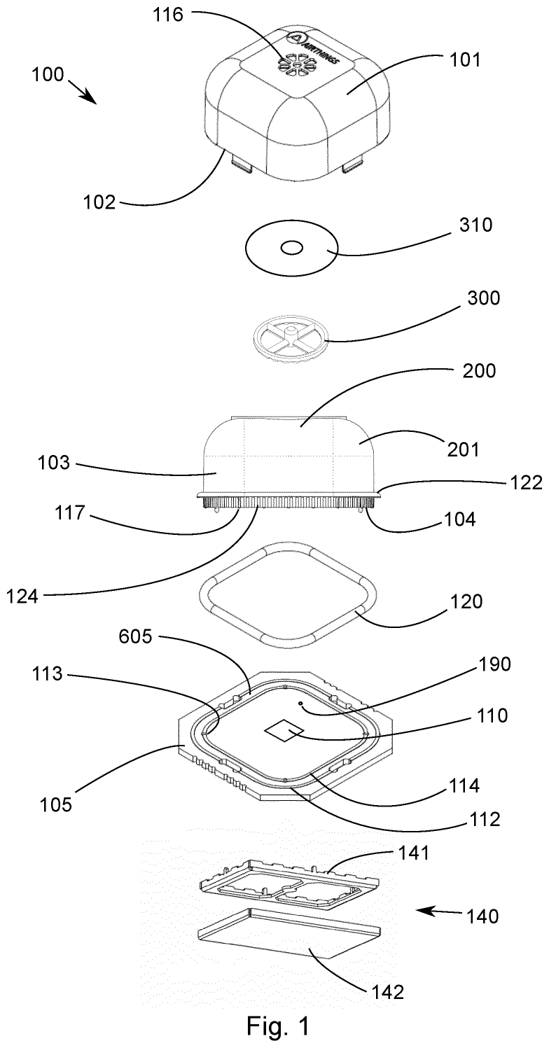

FIG. 1 shows an exploded view of various components of a radon gas sensor;

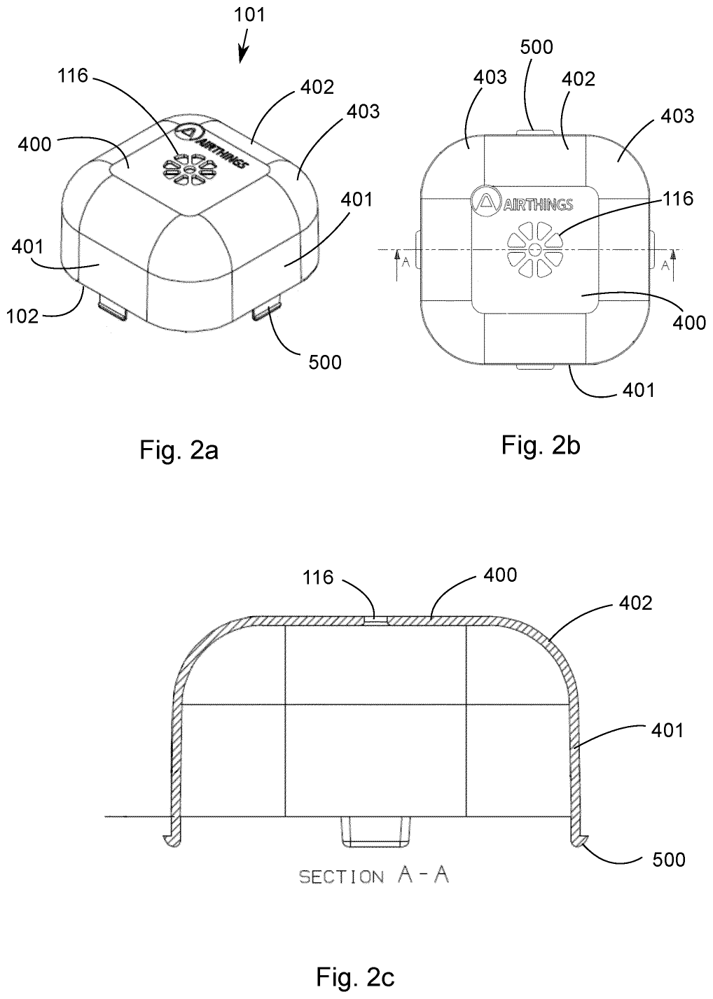

FIGS. 2a, 2b and 2c show an outer dome of a gas sensor;

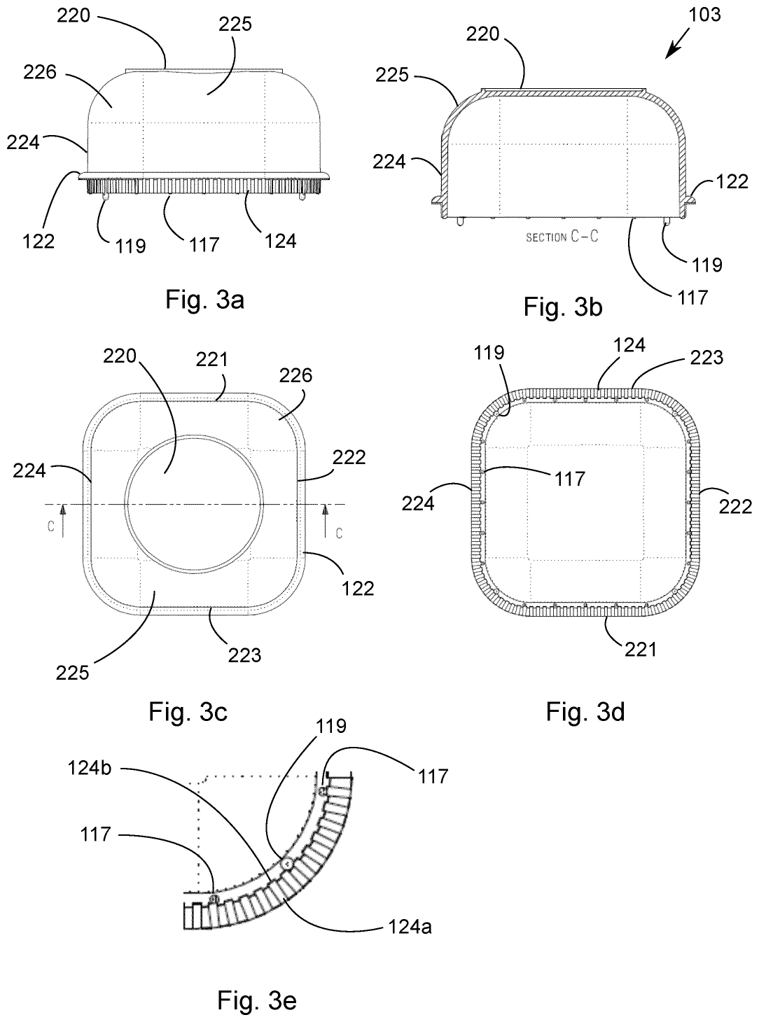

FIGS. 3a — 3e show an inner dome of a gas sensor;

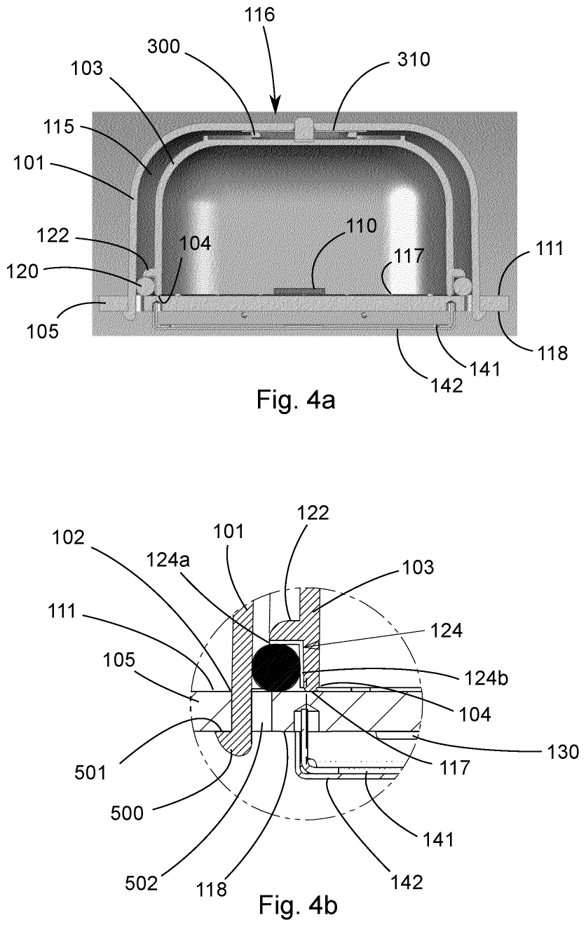

FIG. 4a shows a cross-section of an assembled gas sensor;

FIG. 4b shows an enlarged view of a sealing arrangement;

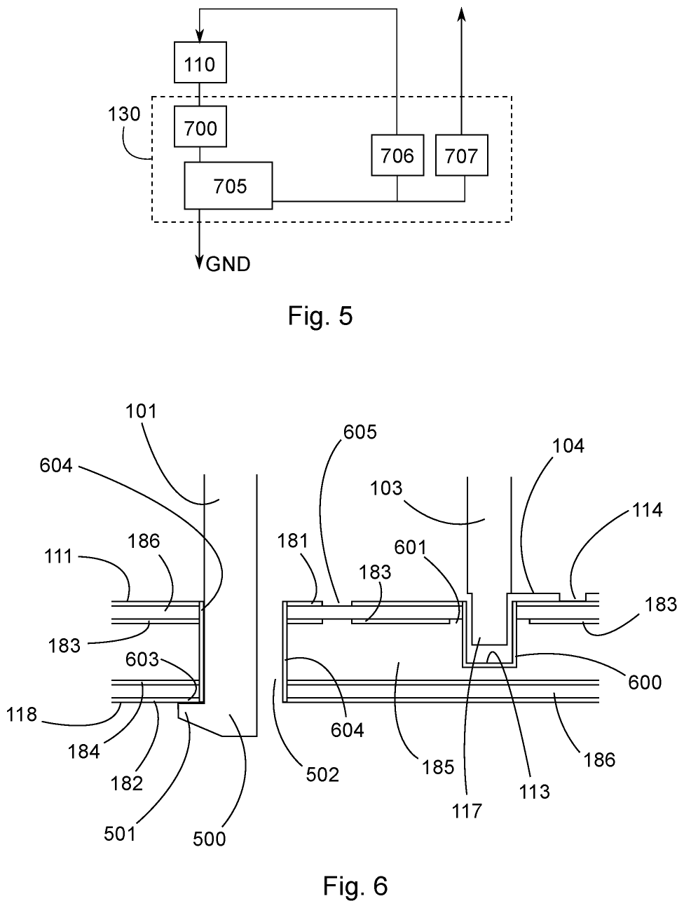

FIG. 5 schematically shows electronics for the gas sensor;

FIG. 6 shows layers in a multilayer printed circuit board; and

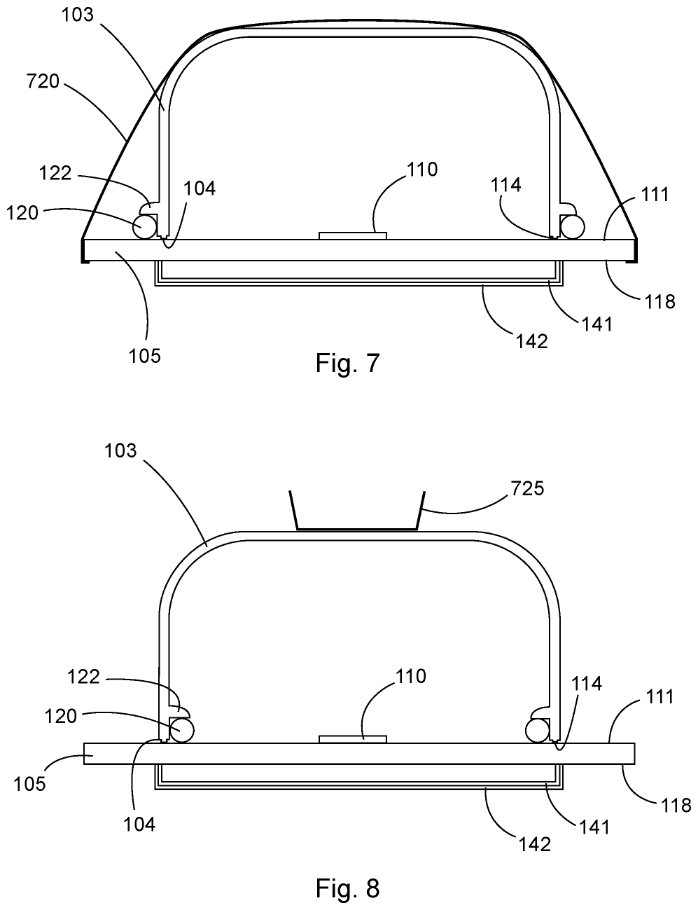

FIGS. 7 and 8 show examples of a single dome gas sensor.

Various components of a radon gas sensor module 100 according to an embodiment of the invention are shown in FIG. 1. These components are shown in an exploded configuration to show their order of assembly, although they are not all shown from the same perspective. These include, an outer dome 101, a spacer 300 and filter 310, an inner dome 103, a gasket 120, a printed circuit board 105 and a faraday cage 140.

The printed circuit board 105 has a photosensor 110 mounted on one side 111 and a hole 190 in its surface conductive layer 181 through which light can pass from a testing device (although the testing device and hole are an optional feature and can be omitted in some embodiments). The hole 190 is only in the surface conductive layer 181 and does not extend through the underlying substrate of the printed circuit board 105 so that it is impermeable to air.

The inner dome 103 is opaque to light and, when mounted on the printed circuit board 105 (specifically by mounting its rim 104 to the conductive trace 114 on the printed circuit board), it forms an opaque chamber. This opaque chamber forms the diffusion chamber of the radon gas sensor 100. Spacers 117 formed on the rim 104 of the inner dome 103 provide a small opening by which air can diffuse underneath the rim 104 and into the interior of the chamber which defines the sensitive volume for the radon gas sensor 100.

The outer dome 101 is mounted over the top of the inner dome 103 and serves as an electromagnetic shield which protects the inner dome 103 from electromagnetic interference. This is particularly important as the inner dome 103 is held at a high voltage. The module 100 is designed for extreme low power operation in order to support long life time on batteries so that the module can be used in a handheld (or at least non-mains powered) device that is easier to position freely without considerations of power supply or needing to change or recharge batteries regularly. The high voltage is supplied by a high-voltage generator with a high output impedance. This high voltage is susceptible to electromagnetic pick-up and in order to avoid such pick-up, the outer dome 101 provides electromagnetic shielding of the inner dome 103. The outer dome 101 is therefore held at a low or ground potential. In addition to providing an electromagnetic shield, the outer dome 101 can be held at a low or ground potential for reason of safety; by having it at ground potential there is less risk of electrical shock when the module is operated without a protective instrument housing. By contrast, leaving the inner dome 103 exposed to users at a potential of around 100V would be less desirable.

In addition to providing the electromagnetic shielding function, the outer dome 101 also forms a diffusion path 115 between an opening 116 in the roof of the outer dome 101 and down between the two domes 101, 103 towards the rim 104 of the inner dome 103. Outer dome 101 is electrically connected to the printed circuit board 105 via its rim 102 contacting conductive trace 112. A gasket 120 located between the inner dome 103 and the outer dome 101 is pressed against the printed circuit board 105 by a lip 122 formed on the outer surface of the inner dome 103. The diffusion path 115 passes over the top of the gasket 120 and down towards the rim 104 between the gasket 120 and the outer surface of the inner dome 103 via air channels 124 formed in the underside of the lip 122 and on the outer surface of the inner dome 103. The gasket 120 seals against the printed circuit board 105, thereby preventing air and light from entering the inner dome 103 under its rim 104 and the gasket 120 seals against the inner surface of the outer dome 101 thereby preventing air from entering the diffusion path 115 other than at the opening 116 in the roof of the outer dome 101.

It will be appreciated that in other embodiments (not illustrated), the lip 122 could be formed on an inner surface of the outer dome 101 while still performing the function of compressing the gasket 120 against the printed circuit board 105 The seal between the gasket 120 and the outer dome 101 could then be on the underside of the lip 122. In other embodiments lips 122 may be provided on both the inner dome 103 and the outer dome 101.

The photosensor 110 is the only electrical component mounted on the first side 111 (seen in FIG. 4b) of the printed circuit board 105 (mounted in a permanent conducting sense, e.g. via soldering or wire bonding). The photosensor 110 is wire bonded to the printed circuit board 105 in a clean room environment so as to avoid unwanted contamination from soldering processes. On the other hand, other electrical components such as processing circuits 130 (indicated in FIG. 5) can be surface mounted on the second (opposite) side 118 of the printed circuit board 105 in a separate process (which may be soldering).

A Faraday cage 140 is provided over at least some of the electrical components 130 on the second side 118 of the printed circuit board 105 to shield them from electromagnetic interference. The Faraday cage 140 shown here is a two part structure comprising a frame 141 which is soldered (surface mounted) onto the second side 118 of the printed circuit board 105 and a cover 142 which attaches to the frame in a separate assembly step. It will be appreciated that the Faraday cage 140 attaches to the underside 118 of the printed circuit board 105 in FIG. 1. When attached to the printed circuit board, the frame 141 is interposed between the cover 142 and the printed circuit board 105.

FIGS. 2a, 2b and 2c show the outer dome 101 in perspective, top view and cross-section respectively. The outer dome 101 in this embodiment has a rounded cuboid shape with a planar roof 400, four side walls 401 perpendicular to the roof 400 and with the edges and corners connecting the roof 400 and walls 401 all being rounded. The rounded edges 402 and rounded corners 403 match the shape of similar structures on the inner dome 103 discussed below so as to form a uniform diffusion path between the inner dome 101 and the outer dome 103.

FIGS. 3a to 3e show various views of an inner dome 103 of the gas sensor 100. FIG. 3a is a side view of the inner dome 103 looking at one side wall 221 of the inner dome 103. FIG. 3b is a cross-section through the inner dome 103. FIG. 3c shows a top view of the inner dome 103 and FIG. 3d shows a view of the inside of the inner dome 103, viewed from the bottom (i.e. looking up at the interior side of the roof 220 of the inner dome 103. The inner dome 103 in this embodiment has a rounded cuboid shape with a planar roof 220 and four side walls 221, 222, 223, 224 perpendicular to the roof 220 and with the edges and corners connecting the roof 220 and walls 221-224 all being rounded. The rounded edges 225 and rounded corners 226 make a more uniform electric field, avoiding the weak spots that can occur in sharp edges and corners. The rounded cuboid shape of the inner dome 103 is the same as that of the outer dome 101 but slightly smaller so as to fit inside the outer dome 101, forming part of the diffusion path 115 between the two domes 101, 103.

As discussed above, the diffusion path 115 ends with air passing under the rim 104 of the inner dome 103 (i.e. between the rim 104 and the printed circuit board 105). With the lip 122 formed on an outer surface of the inner dome 103 (for pressing the gasket 120 against the printed circuit board 105), air must be given a route to bypass the gasket 120 and reach the rim 104. As the gasket 120 seals against the printed circuit board 105, air cannot pass underneath the gasket 120 and therefore a bypass route is provided over the top of the gasket by air channels 124 formed in the underside of the lip 122 and on the outer side of the inner dome 103. Even with the lip 122 compressing the gasket 120 against the printed circuit board 105, the gasket 105 does not deform into the channels 124 to block them. Therefore air can pass around the top and inner side of the gasket 120 and down to the rim 104 of the inner dome 103. The air channels 124 in this embodiment are only 0.5 mm wide such that they provide a very narrow constriction through which the air must pass, thereby encouraging plate-out of any aerosols present in the air.

The rim 104 of the inner dome 103 is provided with a number of spacer projections 117 that extend a short distance (in this embodiment about 0.15 mm) down from the rim 104 towards the printed circuit board 105. These spacer projections 117 ensure that, even when the inner dome 103 is biased into contact with the printed circuit board 105, there remains a small gap underneath the rim 104 by which air can diffuse into the interior of the inner dome 103 (i.e. into the diffusion chamber which is the sensitive volume for the gas sensor 100).

Also shown in FIGS. 3a, 3b, 3d and 3e are four locating pins 119 formed on the rim 104 of the inner dome 103 much like the spacer projections 117, but longer. The locating pins 119 are arranged to fit into corresponding locating recesses 113 (seen in FIGS. 1 and 6) in the upper surface 111 of the printed circuit board 105 so as to ensure alignment of the inner dome 103 and outer dome 101 with the corresponding conductive traces 112, 114 on the printed circuit board 105 and also to facilitate holding the inner dome 103 and gasket 120 during a mounting process of the outer dome 101. The locating recesses 113 in the printed circuit board 105 are deeper than the locating pins 119 so that the locating pins 119 do not contact the bottom of the recesses 113. This ensures that the spacer projections 117 are not prevented from contacting the conductive trace 114 and that the gap under the rim 104 is defined by the height of the spacer projections 114. More specifically, the difference between the length of the locating pins 119 and the length of the spacer projections 117 (i.e. the length that the locating pins 119 project below the surface of the printed circuit board 105) is less than the depth of the locating recesses 113 so that the locating pins 119 will not reach the bottom of the locating recesses 113.

Where the printed circuit board 105 is a multilayer printed circuit board with both surface conductive layers 181, 182 and internal conductive layers 183, 184 as shown in FIG. 6, the depth of the locating recesses 113 is generally greater than the depth of the first internal conductive layer 183 of the printed circuit board 105 (i.e. the one closest to the surface). Therefore in such cases, the locating recesses 113 will project down through at least one internal conductive layer 183. In order to preserve the faraday shielding formed by the surface conductive layer 181 (discussed further below), the locating recesses 113 are provided with a lining 600 of electrically conductive material. As this lining 600 projects through the internal conductive layer 183 (or several such internal conductive layers), the internal conductive layer 183 has an insulating region 601 around the locating recess 113 so as to avoid electrical connection between the two layers 181, 183. This insulating region 601 may be formed simply by removing part of the internal conductive layer 183 during manufacture of the printed circuit board 105.

FIG. 3e shows an enlarged view of the lower right corner of FIG. 3d, showing the spacer projections 117, a locating pin 119 and air channels 124. The channel 124 is shown here as comprising two parts: a first part 124a which lies on the underside of the lip 122 and passes over the top of the gasket 120, and a second part 124b (which connects with the first part 124a so that air can flow from one to the other) which extends along the outside surface of the inner dome 103 from the lip 122 down to the rim 104 and passing behind the gasket 120 (between the gasket 120 and the inner dome 103).

FIG. 4a shows a cross-section through the assembled structure of FIG. 1. FIG. 4b shows an enlarged view of the sealing arrangement on the left hand side of FIG. 4a. In particular, it can be seen clearly in FIG. 4b that the gasket 120 is pressed by lip 122 into contact with the upper surface 111 of printed circuit board 105. This creates a seal between the gasket 120 and the printed circuit board 105 which prevents both air and light from passing underneath the gasket 120. The blocking of air at this point is important so as to avoid a bypass of the diffusion path 115 that is created between the opening 116 of the outer dome 101 and the rim 104 of the inner dome 103. The blocking of light is also important as it is possible that a small amount of light may enter underneath the rim 102 of the outer dome 101 as discussed below (note that the rim 102 is not directly visible in FIG. 4b as the cross-section passes through the clip 500, but its position is indicated by reference 102). The gasket 120 also seals against the inside surface of the outer dome 101, again preventing a bypass into the diffusion path 115 over the gasket 120.

It will be appreciated that in order to compress the gasket 120 against the printed circuit board, a force must be supplied to push the lip 122 towards the printed circuit board 105. This may be provided by any mechanism that holds the inner dome 103 in place against the printed circuit board 105. However, in order to avoid the use of permanent fixing mechanisms such as screws or glue, the inner dome 103 in this embodiment is pressed against the printed circuit board by the outer dome 101 which acts on the spacer 300 that is interposed between the roof of the inner dome 103 and the roof of the outer dome 101 and sized so as to contact both domes 101, 103 and thereby transmit force from one to the other. This contact also holds the filter paper 310 between the spacer 300 and the roof of the outer dome 101, trapping it therebetween and ensuring that air must pass through the filter paper 310 in order to enter the diffusion path 115 and thereby reducing the number of larger particles entering the diffusion path 115. The force that holds the outer dome 101 in place is provided by clips 500 that pass through holes 502 in the printed circuit board 105 and spring out to contact (and hold against) the underside 118 of the printed circuit board 105 via an extension 501 of the clip 500. To hold the gasket 120 in the compressed and sealed state, the outer dome 101 is pressed down onto the spacer 300 and the inner dome 101 so as to compress the gasket 120 and at the same time, the clips 500 pass through the holes 502 and the extensions 501 such that they clip under and hold against the printed circuit board 105 while the gasket 120 is in the compressed state. The gasket 120 provides a reaction force that pushes away from the printed circuit board 105 against lip 122 of the inner dome 103. To keep the inner dome 103 in electrical contact with the printed circuit board 105, this force must be countered by the downward force from the outer dome 101 which is provided by the clips 500. Some relative movement of the inner dome 103 and outer dome 103 is accommodated by flexing of the roofs of the inner dome 103 and outer dome 101 either side of the spacer 300. As the outer dome 101 is pushed upwards to bring the clips 500 into contact with the underside 118 of the printed circuit board 105, the rim 102 of the outer dome 101 may be lifted very slightly away from the upper side 111 of the printed circuit board 105. This allows the possibility of light and air to enter underneath the rim 102, but any such light or air is then blocked by the gasket 120 sealing against the printed circuit board 105 and the inner surface of the outer dome 101.

The separation of the rim 102 of the outer dome 101 from the upper side 111 of the printed circuit board 105 also affects the reliability of electrical connection being made via the rim 102. Therefore in this embodiment the clips 500 (including the extension 501) are conductive and are arranged to contact a conductive trace on the underside 118 of the printed circuit board 105. The connection here is reliable as the outer dome 101 is biased upwards away from the upper surface 111 of the printed circuit board 105, biasing the extensions 501 into firm contact with the underside 118 of the printed circuit board 105. The outer dome 101 may be made from metal or it may be coated with a conductive material. In this example, the outer dome 101 is made from metallised plastic. For added reliability of electrical connection, the clips 500 can also be arranged to contact a wall of the hole 502. The wall of the hole 502 can also have a conductive liner 604 so that electrical contact is made with the conductive clip 500. The clip 500 may be sprung so that it is biased against the liner 604.

The arrangement of a clip 500 and extension 501 in electrical contact with a conductive layer 182 on the underside 118 of the printed circuit board 105 is shown in FIG. 6 (it will be appreciated that the gasket 120 is omitted from this figure for clarity). FIG. 6 shows the construction of a multilayer printed circuit board 105 with a core substrate 185 (typically formed from “FR4” glass-fibre reinforced polymer) having two internal conductive layers 183, 184 formed thereon, then two layers of prepreg material (typically also glass-fibre reinforced polymer), then two outer surface conductive layers 181, 182. Each of the layers 181, 182, 183, 184 may be etched or otherwise shaped to form conductive pads and/or traces for interconnecting various components. In addition, electrical vias may be formed between layers 181, 182, 183, 184 in known manner for interconnecting those layers.

FIG. 6 also illustrates the faraday cages that may be formed with this construction. One faraday cage may be formed by the inner dome 103, electrically connected to the surface conductive layer 181 via the spacer projections 114. As the first surface conductive layer 181 may be substantially continuous and as the inner dome 103 is electrically conductive (either being formed from metal or metallised plastic or the like), together they form a faraday cage around the photosensor 110 as well as providing a high voltage surface for forming an electric drift field.

The outer dome 101 is electrically connected to a contact pad 603 on the second surface conductive layer 182 on the underside 118 of the printed circuit board 105. As it is convenient to surface mount other components to this second conductive layer 118, the contact pad 603 may be connect by a via (not shown) to one of the internal conductive layers 183, 184 which extend underneath the surface conductive layer 181 and the photodiode 110 while the outer dome 101 extends around and over the inner dome 103 so that together they form a faraday shield around the inner dome 103 that can be held at a low (or ground) voltage to shield the high voltage inner dome 103 from electromagnetic interference. An insulating gap 605 is formed in the surface conductive layer 181 so as to isolate the high voltage connection to the inner dome 103 from the low voltage connection to the outer dome 101. In the embodiment shown in FIG. 6, all four conductive layers 181, 182, 183, 184 are bridged by the conductive liner 604 so that all four layers are at the same potential (ground potential in this embodiment) in the region of the hole 502, although other parts of those conductive layers 181, 182, 183, 184 are of course isolated from this region. In addition, electronics for the control and signal processing may be surface mounted on the underside 118 of the printed circuit board on surface conductive layer 182. The faraday cage 140 may also be mounted to this surface conductive layer 182 to cover those components and protect them from electromagnetic interference. The faraday cage 140 may be connected by a via to an internal conductive layer 183, 184 to complete a full electrical surround of the processing circuits. It will be appreciated that it is particularly convenient to use the internal conductive layer 184 to connect to the faraday cage 140 and the internal conductive layer 183 to connect to the outer dome 101 so that all faraday cages are electrically separate.

FIG. 5 schematically shows the electronics 130, including amplifying circuit 700 that receives the output signal from photosensor 110. The signals from amplifying circuit 700 feed into microprocessor 705 where the data can be processed. Microprocessor 705 also generates bias voltages for the photosensor 110 and the inner dome 103. These bias voltages will typically be of much greater magnitude than the operating voltage of the microprocessor which typically operates at around 3-5 V. The larger magnitude voltages may be generated by any suitable voltage conversion or boosting circuit. In FIG. 5 these are schematically illustrated as a photosensor bias circuit 706 which generates a bias voltage (e.g. of around −70 V) and applies it to the photodiode 110, and a diffusion chamber bias circuit 707 which generates a bias voltage (e.g. of around 100 V) and applies it to the inner dome 103. With the example voltages given here, the electric field between the inner dome 103 and the photosensor 110 is generated by a voltage difference of 170 V and with a distance between inner dome 103 and photodiode 110 in the region of 1.5-2.5 cm, can create an electric field with strength in the region of 60 to 120 V/cm. The microprocessor 705 (or indeed other parts of the electronics 130) may also output a ground connection (GND) that can be connected to the outer dome 101, thereby providing a safe surface for user contact and an electromagnetic shield for the inner dome 103.

FIGS. 7 and 8 illustrate two alternative radon gas monitors in which only a single dome 103 is used (here most equivalent to the inner dome 103 discussed above as it does not contain an opening in its roof and is spaced from the printed circuit board 105 by spacer projections 114). FIG. 7 shows an example in which the gasket 120 is held against the printed circuit board 105 by lip 122 in much the same way as it is in FIGS. 4a and 4b, although in FIG. 7 the gasket 120 only forms a seal against the surface 111 of the printed circuit board 105 (with a diffusion path formed between the gasket 120 and the lip 122 and outer wall of the dome 103 by channels 124 (not visible in FIG. 7, but with the same structure as discussed above). Here the diffusion path is much shorter as it is only formed by the path around the gasket 120, but the gasket 120 is still held firmly against the printed circuit board 105 so as to prevent air from entering under the rim 104 by any path other than the diffusion path. Light also cannot enter via this diffusion path due to its convoluted nature. To aid with light absorption the gasket 120 is black (also the case in FIGS. 1-6).

FIG. 8 shows an alternative single dome arrangement similar to that of FIG. 7 but with the lip 122 formed on the inside of the dome 103. The diffusion path in this instance is first under the rim 104 (between spacer projections 114), then over the gasket 120 and under the lip 122 (again via channels 124 as discussed above). In the case where the photosensor 110 is a photodiode and an electric drift field is set up inside the dome 103, the presence of the lip 122 inside the dome 103 has an effect on the strength and uniformity of the drift field, but such effects would have less impact where the photosensor 110 is a photomultiplier. This arrangement has benefits in terms of overall area of the sensor.

FIGS. 7 and 8 also show two different ways of holding the dome 103 against the printed circuit board 105. In FIG. 7 a tension strap 720 is attached over the ends of the printed circuit board 105 on either side of the dome 103 and is wrapped over the top of the dome 103 so as to provide a biasing force on the dome 103 towards the printed circuit board 105, thereby ensuring electrical contact. In FIG. 8, an external structure 725 presses down on the dome 103 so as to hold the dome 103 firmly against the printed circuit board 105. Such a structure 725 may be provided for example on an internal surface of an instrument housing. It could be a rigid structure (e.g. moulded into the housing) or it could be a compressible structure (e.g. a piece of foam or rubber) attached to another external structure. It will be appreciated that tension strap 720 and external structure 725 are not specific to these embodiments, but are interchangeable and can also be used in the embodiments of FIGS. 1 to 6 as well.

It will be appreciated that many variations of the above embodiments may be made without departing from the scope of the invention which is defined by the appended claims.

1. A sensor, comprising:
a printed circuit board;
a detector mounted on the printed circuit board;
an inner dome that is electrically conductive and is mounted on the printed circuit board so as to form a diffusion chamber around the detector; and
an outer dome that is electrically conductive and is mounted on the printed circuit board, surrounding the inner dome.

2. A sensor as claimed in claim 1, wherein the sensor is arranged to apply a first voltage to the inner dome and a second voltage to the outer dome.

3. A sensor as claimed in claim 2, wherein the first voltage has a magnitude greater than that of the second voltage.

4. A sensor as claimed in claim 3, wherein the second voltage is ground.

5. A sensor as claimed in claim 1, wherein the sensor is arranged to apply a detector bias voltage to the detector.

6. A sensor as claimed in claim 1, wherein the inner dome is connected to a first conductive layer of the printed circuit board so as to form a faraday shield around the detector.

7. A sensor as claimed in claim 1, wherein the outer dome is connected to a second conductive layer of the printed circuit board so as to form a faraday shield around the inner dome.

8. A sensor as claimed in claim 1, wherein the inner dome and the outer dome are substantially the same shape and concentrically arranged.

9. A sensor as claimed in claim 1, wherein the inner dome has a rounded shape.

10. A sensor as claimed in claim 9, wherein the inner dome has a rounded cuboid shape with rounded edges and corners.

11. A sensor as claimed in claim 1, wherein a diffusion path for air exchange with the interior volume of the inner dome passes between the inner dome and the outer dome.

12. A sensor as claimed in claim 11, wherein an entrance to the diffusion path is located centrally in a roof of the outer dome.

13. A sensor as claimed in claim 1, further comprising a gasket arranged to seal against a surface of the printed circuit board.

14. A sensor as claimed in claim 13, wherein the gasket is biased against the printed circuit board by a lip formed on at least one of the inner dome and the outer dome.

15. A sensor as claimed in claim 13, wherein the gasket is located between the inner dome and the outer dome.

16. A sensor as claimed in claim 15, wherein the gasket seals against an inner surface of the outer dome.

17. A sensor as claimed in claim 13, wherein the gasket seals against an outer surface of the inner dome except that one or more air channels are formed to bypass the gasket and are formed along the outer surface of the inner dome, connecting with a rim of the inner dome adjacent to the printed circuit board.

18. A sensor as claimed in claim 17, wherein the gasket is biased against the printed circuit board by a lip formed on the inner dome and wherein the one or more air channels each extends along the underside of the lip.

19. A sensor as claimed in claim 1, wherein a biasing member is provided to bias the outer dome towards the printed circuit board and to ensure electrical contact of the outer dome with the printed circuit board.

20. A sensor as claimed in claim 19, wherein the biasing member comprises one or more clips provided on the outer dome that extend through holes in the printed circuit board and engage with a side of the printed circuit board opposite the side on which the outer dome is located.

21. A sensor as claimed in claim 19, wherein the outer dome is arranged to bias the inner dome into electrical contact with the printed circuit board.

22. A sensor as claimed in claim 21, wherein a spacer is provided between the outer dome and the inner dome so as to transmit a biasing force from the outer dome to the inner dome.

23. A sensor as claimed in claim 22, wherein an entrance to a diffusion path is located centrally in a roof of the outer dome, and wherein the spacer forms a ring around the entrance and has one or more holes or channels formed in its side wall to allow air to flow from the entrance along the diffusion path.

24. A sensor as claimed in any claim 1, wherein one of the inner dome and the outer dome comprises one or more locating pins extending towards the printed circuit board and wherein the printed circuit board has a corresponding one or more locating recesses formed therein to receive the one or more locating pins, and wherein the one or more locating recesses are sufficiently deep that the one or more locating pins do not contact the bottom of the one or more recesses.

25. A sensor as claimed in claim 24, wherein the one or more locating pins are formed on the inner dome, wherein the inner dome comprises one or more spacer projections formed on the rim and extending towards the printed circuit board, and wherein the depth of each of the one or more recesses is greater than the difference between the length of the corresponding locating pin and the length of the spacer projections.

26. A sensor as claimed in claim 24, wherein the printed circuit board is a multilayer printed circuit board comprising a surface conductive layer, portions of which are in contact with the inner dome and the outer dome, and an internal conductive layer located at a first depth below the surface conductive layer, wherein the depth of the one or more locating recesses is greater than the first depth and wherein the internal conductive layer comprises an insulating region around each of the one or more locating recesses.

27. A sensor as claimed in claim 24, wherein each of the one or more locating recesses is lined with electrically conductive material.

28. A sensor, comprising:
a printed circuit board;
a detector mounted on the printed circuit board;
a dome that is electrically conductive and is mounted on the printed circuit board so as to form a diffusion chamber around the detector; and
a gasket arranged to seal against a surface of the printed circuit board;
wherein the gasket is biased against the printed circuit board by a lip formed on the dome.

29. A sensor as claimed in claim 28, wherein the lip extends from an outer surface of the dome.

30. A sensor as claimed in claim 28, wherein the gasket seals against an outer surface of the dome except that one or more air channels are formed to bypass the gasket and are formed along the outer surface of the dome, connecting with a rim of the dome adjacent to the printed circuit board.

31. A sensor as claimed in claim 30, wherein the one or more air channels each extends along the underside of the lip.

32. A sensor, comprising:
a printed circuit board;
a detector mounted on the printed circuit board;
a dome that is electrically conductive and is mounted on the printed circuit board so as to form a diffusion chamber around the detector; and
wherein the dome comprises one or more locating pins extending towards the printed circuit board and wherein the printed circuit board has a corresponding one or more locating recesses formed therein to receive the one or more locating pins, and wherein the one or more locating recesses are deeper than the length of the one or more locating pins.

33. A sensor as claimed in claim 32, wherein the dome comprises one or more spacer projections formed on the rim and extending towards the printed circuit board, and wherein the depth of each of the one or more recesses is greater than the difference between the length of the corresponding locating pin and the length of the spacer projections.

34. A sensor as claimed in claim 32, wherein the printed circuit board is a multilayer printed circuit board comprising a surface conductive layer, portions of which are in contact with the dome, and an internal conductive layer located at a first depth below the surface conductive layer, wherein the depth of the one or more locating recesses is greater than the first depth and wherein the internal conductive layer comprises an insulating region around each of the one or more locating recesses.

35. A sensor as claimed in claim 32, wherein each of the one or more locating recesses is lined with electrically conductive material.
